(19) United States
(12) Patent Application Publication
(10) Pub. No.: US 2006/0089536 A1
(43) Pub. Date: Apr. 27, 2006

(54) Expandable surgical retractor system for minimal access surgery

(75) Inventors: Mick J. Perez-Cruet, Bloomfield, MI (US); Leonel Dominguez, Jacksonville, FL (US); Shahram Shaun Zamani, Tehran (IR)

Correspondence Address: AKERMAN SENTERFITT, P.O. BOX 3188, WEST PALM BEACH, FL, 33402-3188, US

(73) Assignee: U.S. SPINAL TECHNOLOGIES, LLC, Boca Raton, FL (US)

(21) Appl. No.: 10/973,501

(22) Filed: Oct. 26, 2004

(51) Int. Cl. A61B 1/32

(52) U.S. Cl. 600/210

(57) ABSTRACT

A retractor for surgery includes a retractor body and an elongated and expandable structure attached to the retractor body. The expandable structure, when expanded, has an elongated open interior for the insertion of a portion of a surgical instrument. Drive structure is provided for expanding the expandable structure. A retractor system and method for retracting an incision are also disclosed.

### CROSS-REFERENCE TO RELATED APPLICATIONS

Not applicable.

### BACKGROUND OF THE INVENTION

### FIELD OF THE INVENTION

This invention relates generally to surgical instruments, and more particularly to retractor instruments and methods.

Minimal access surgery or percutaneous surgery utilizes a small skin incision approximately 10 mm long. A muscle-tissue dilator is then inserted through the incision in order to further expand the wound to about 24-32 mm in diameter to facilitate the insertion of surgical instrumentation and implants. The muscle-tissue dilator is a surgical retractor that reduces the need for large, open incisions, tissue scarring and pain.

Prior methods and devices for retracting incisions utilize a series of rigid tubes of varying diameters and lengths. Each tube is sequentially-sized of increasing diameter such that one can be fitted over the other and individually pushed through the incision, fascia, and muscle bed toward the surgical site of interest. The tubes cannot be pushed too deeply, as they may strike vital structures such as the spinal cord, nerves, or blood vessels. The potential for improper placement and manipulation of the tubes may be dangerous and commonly requires x-ray imaging during each successive tube placement to verify tube position. As only a portion at the end of the tube serves to pry open tissue during placement, strong muscle resistance acts to expulse the tube, which sometimes results in the necessity to reposition the tube. This process is prone to placement error, dangerous manipulation, increased radiation exposure, and additional surgical time. It would therefore be desirable to provide a retractor device and method which save time and do not require repetitive manipulation, avoid undue force in placement in the incision, require less x-ray (radiation) exposure to the patient and medical staff, and which will effectively retain a proper position in the incision.

### SUMMARY OF THE INVENTION

A retractor for surgery includes a retractor body and an elongated and expandable structure attached to the retractor body. The expandable structure, when expanded, has an elongated open interior for the insertion of a portion of a surgical instrument. Drive structure is provided for expanding the expandable structure.

The expandable structure can have any suitable construction. In one aspect, the expandable structure is an elongated coil. The drive structure expands and retracts the diameter of the coil. The drive structure can be any suitable structure. In one aspect, the drive structure comprises gears which engage the coil at drive surfaces.

The expandable structure in another aspect comprises at least first and second portions. The drive structure moves the first and second portions relative to one another. The first and second portions can be curved or substantially C-shaped (semi-circular or elliptical) in cross-section.

The drive structure can be moveable within the retractor body or can be external to the retractor body and lockable in position to maintain retractor body securely in a desired position unless unlocked to resume opening or closure of retractor body. A K-wire can be provided to assist in positioning the device.

In another aspect, the expandable structure comprises retractor arms. The drive structure is operable to expand the retractor arms.

A retractor system for surgery comprises a retractor body having elongated and expandable structure attached to the retractor body. The expandable structure, when expanded, has an elongated open interior space for the insertion of a surgical instrument. Drive structure is operable to expand the expandable structure. At least one surgical instrument is positionable in the open interior space. These include bone drill bits, curettes, probes, endoscopes, cautery devices, as well as other instruments.

A method for retracting an incision includes the steps of providing a retractor comprising a retractor body with elongated and expandable structure attached to the retractor body. The expandable structure, when expanded, has an elongated open interior space for the insertion of a surgical instrument. Drive structure is provided for expanding the expandable structure. The retractor is inserted into the incision. The drive structure is then operated to expand the expandable structure. A surgical instrument can then be inserted at least in part through the open interior space of the expandable structure.

### BRIEF DESCRIPTION OF THE DRAWINGS

There is shown in the drawings embodiments which are presently preferred, it being understood, however, that the invention can be embodied in other forms without departing from the spirit or essential attributes thereof, wherein:

FIG. 1 is a perspective view of a retractor system according to the invention.

FIG. 2 is an exploded view of a retractor system according to the invention.

FIG. 3 is a perspective view of a retractor system in a closed position with transmission gear cover removed for visualization of the drive mechanism.

FIG. 4 is a perspective view of a retractor system in the open position.

FIG. 5 is a perspective view of a retractor according to the invention in an open position.

FIG. 6 is a cross-section taken along A-A in FIG. 5.

FIG. 7 is a cross-section taken along B-B in FIG. 5.

FIG. 8 is a perspective view of a retractor according to an alternative embodiment.

FIG. 9 is a cross-section of expandable structure according to an alternative embodiment.

FIG. 10 is a side elevation, partially broken away and partially in phantom, of drive structure in a first position.

FIG. 11 is a side elevation of drive structure in a second position.

FIG. 12 is a perspective view, partially broken away and partially in phantom, illustrating a retractor according to an alternative embodiment.

### DETAILED DESCRIPTION OF THE INVENTION

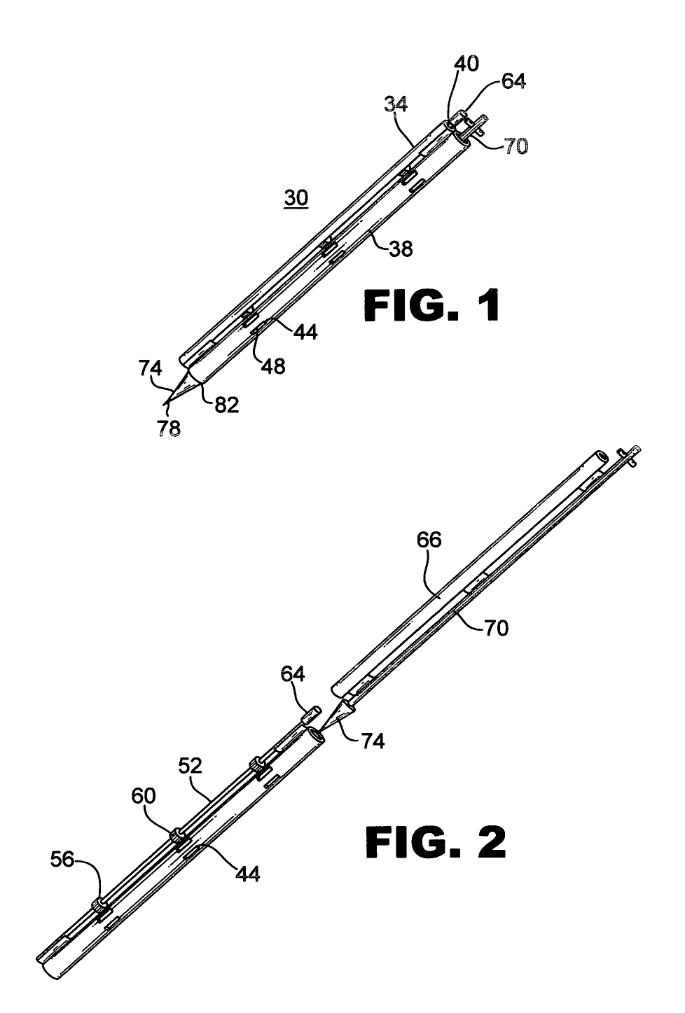

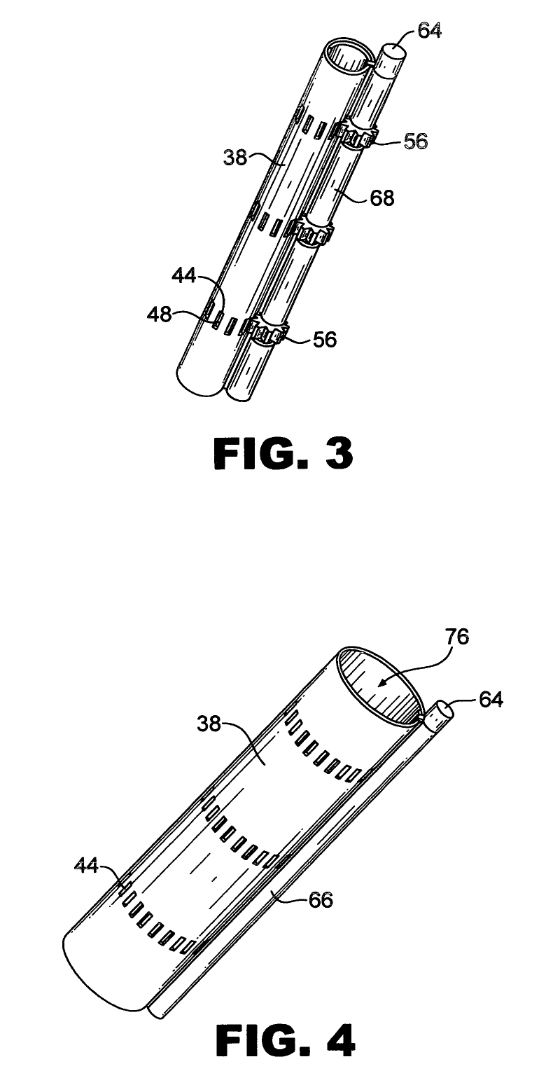

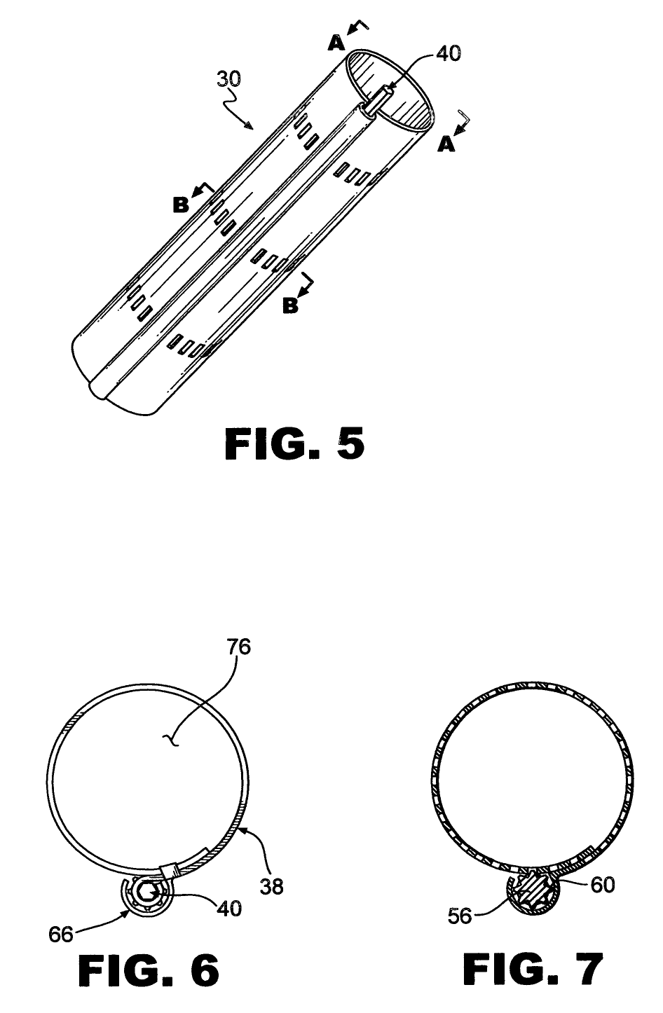

There are shown in FIGS. 1-7 an embodiment of a retractor 30 having expandable structure 38 and drive structure 40. The expandable structure 38 can have any suitable shape, but is at least in part elongated and, when in the open position, has an open interior space into which can -be positioned a surgical instrument. The expandable structure 38 shown in FIGS. 1-7 is an elongated coil, although other expandable structures are possible. The elongated coil 38 connects to the drive structure 40 such that the drive structure 40 is operable to expand the coil from a closed position, shown in FIG. 3, to an open position shown in FIG. 4.

The manner in which the drive structure 40 expands the expandable structure 38 can be varied. In the embodiment shown in FIGS. 1-7, the elongated coil 38 includes slots 44 defining drive surfaces 48. The drive structure 40 includes an elongated shaft 52 (FIG. 2) having gears 56 having gear teeth 60 which engage the slots 44. Suitable structure such as a drive head 64 is provided for engaging and rotating the shaft 52. In this manner, the teeth 60 of gears 56 are moved within slots 44 and engage the drive surfaces 48 to expand the coil 38 from the closed position to the open position. In a reverse direction of rotation, the action of the drive structure 40 retracts the coil from the open position to the closed position. A cover 66 can be positioned over the drive shaft 52 to prevent tissues from entering the drive structure.

The device can be made by any suitable process. In one process, the elongated coil 38 is manufactured from a planar sheet of material. Locking tabs 68 are folded over the elongated shaft 52 between the gears 56 so as to secure the shaft 52 to the coil. The opposite end of the planar sheet is then rolled into a coil and the slots 44 engaged to the teeth 56 to hold the sheet in the coiled position. The cover 66 can then be positioned over the shaft 52 and tabs 68.

Operation of the drive structure causes the coil 38 to unwind from the closed position shown in FIG. 3 to the open position shown in FIG. 4-7. As shown in FIG. 4, the expanded diameter defines an open interior space 76 into which a suitable surgical instrument can be inserted, such as bone drill bits, curettes, probes, suction, cautery, endoscope and other surgical instruments.

Other structure can be provided with the retractor. There is shown in FIGS. 1-2 a K-wire 70 which passes through a head 74. The head 74 can rest or temporarily attach onto an edge 82 of the coil 38 when the coil 38 is in the closed position, as shown in FIG. 1. The K-wire 70, as is known in the art, has a positioning point 78 which is driven into the bone or soft tissue in order to guide the device and possibly other surgical devices. Expansion of the coil 38 after insertion permits the removal of the K-wire and/or head 74 as desired. The head 74 can have a suitable bore through which the K-wire 70 is mounted, such that the head 74 can be removed while the K-wire 70 remains in place to guide surgical instruments.

There is shown in FIG. 8 an alternative embodiment 100 in which drive structure 104 is provided and connected to expandable structure 108. The expandable structure 108 is comprised of a first section 114 and second section 118 which are semi-cylindrical and mated. Operation of the drive structure, as by operation of a drive head 128, causes rotation of the shaft 104 which causes the sections 114, 118 to slide past one another from a closed position to an open position. Locking structure can be provided to prevent the ends of the sections 114, 118 from moving past one another. These can be interlocking hooked portions or a groove and protrusion configuration.

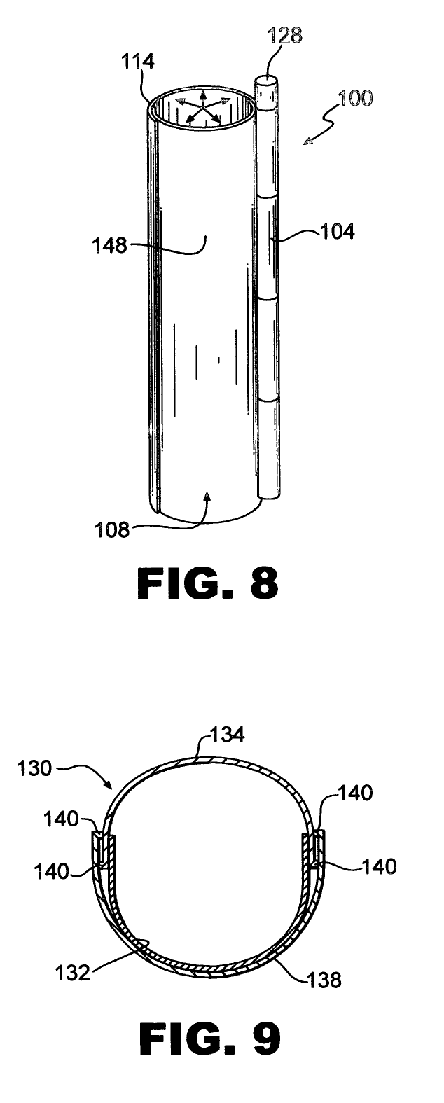

There is shown in FIGS. 9-11 an alternative embodiment. In this embodiment, expandable structure 130 comprises a fixed internal tube 132 and mated sections 134, 138, which are generally semi-spherical in cross-section as shown. The sections 134 and 138 can move past one another. Protruding ends 140 prevent the sections 134 and 138 from becoming disengaged.

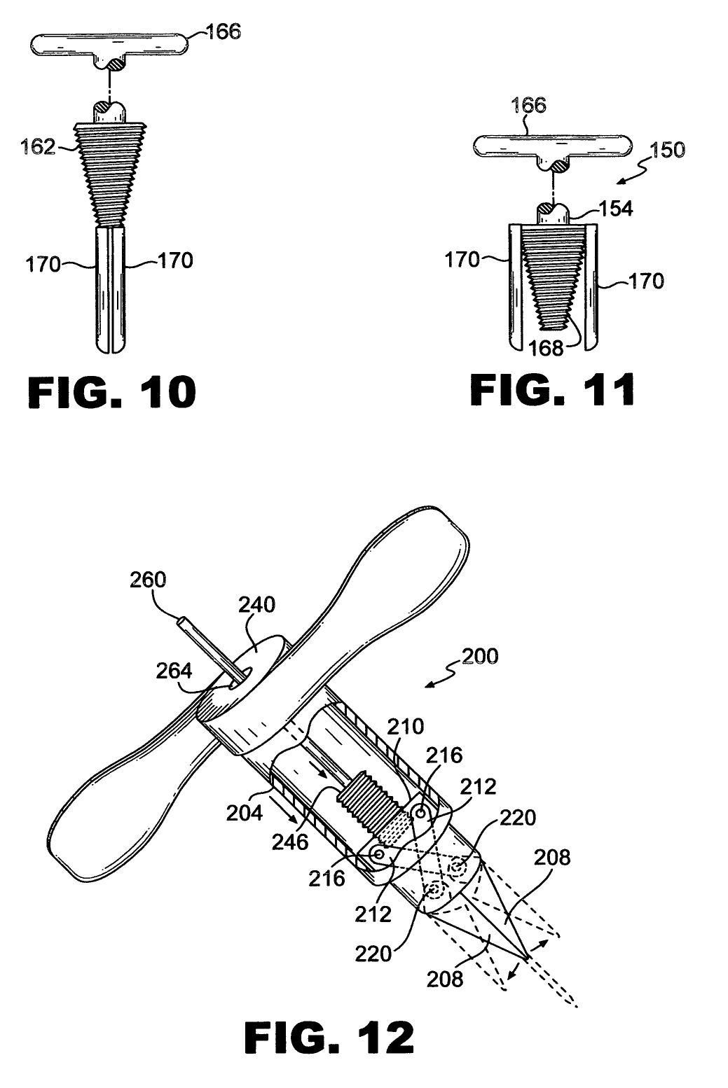

Drive structure is provided to drive the sections 134 and 138 of the expandable structure 130 apart from a closed to an open configuration. Any suitable drive'structure is possible. There is shown in FIG. 10 a drive structure having a drive wedge 162 and arms 170. Operation of the wedge 162 is by a T-handle 166 which causes threads 168 to advance the drive wedge 162 and open drive arms 170 from the closed position shown in FIG. 10 to the open position shown in FIG. 11. This action will cause the first section 134 to move away from the section 138 such that the expandable structure 130 moves from the open position to the closed position.

There is shown in FIG. 12 an alternative embodiment 200. In this embodiment, a elongated body 204 has expandable structure comprising arms 208. The arms 208 are joined to a screw block 210 by members 212. The members 212 are pivotally connected at connections 216 to the drive block 210 and by pivotal connections 220 to the arms 208. A drive handle 240 is provided and operated by a threaded member 246. Rotation of the handle 240, and thereby threads 246, causes advancement of the screw block 210 and opening of the expandable arms 208 from the closed position shown in FIG. 12 to the open position shown by phantom lines in FIG. 12. A K-wire 260 can be threaded through an opening 264 to assist in positioning of the device. The device can be removed while the K-wire 260 remains in place.

According to a method of the invention, a retractor according to the invention is positioned in an incision. Drive structure is operated to expand elongated expandable structure from a closed position to an open position. This expansion can incremental or be substantially continuous, such that the expandable structure can be expanded to any of a plurality of different dimensions. An open interior within the expandable structure provides the insertion of a surgical instrument. Also, an access port tube structure as can be positioned within or over the expandable structure to permit the retractor to be removed. The tube will retain the incision in the retracted position, and the problems of the prior art resulting from the insertion and removal of multiple differently dimensioned tubes can be avoided.

The retractor of the invention can be manufactured from various materials, including disposable plastics and metals. Also, the retractor can be made of a stainless steel which can be autoclaved and reused. The retractor and expandable structure can be provided in differing dimensions. In one embodiment, the expandable structure is between about 40 mm and 180 mm long and can expand from a closed position of about 4 mm to 8 mm diameter to an expanded dimension of about 24 mm to 32 mm diameter.

This invention can be embodied in other forms without departing from the spirit or essential attributes thereof and, accordingly, reference should be had to the following claims rather than the foregoing specification as indicating the scope of the invention.

1. A retractor for surgery, comprising:
a retractor body;
elongated and expandable structure attached to said retractor body, said expandable structure, when expanded, having an elongated open interior for the insertion of a surgical instrument; and
drive structure operable to expand said expandable structure.

2. The retractor of claim 1, wherein the expandable structure is an elongated coil.

3. The retractor of claim 2, wherein the drive structure expands and retracts the coil, and locks against movement until unlocked.

4. The retractor of claim 3, wherein the drive mechanism comprises gears which engage the coil at drive surfaces.

5. The retractor of claim 1, wherein the expandable structure comprises at least first and second portions; said drive structure moving said first and second portions relative to one another.

6. The retractor of claim 1, wherein said drive structure is moveable within said retractor body.

7. The retractor of claim 1, wherein said retractor comprises structure for engaging a K-wire.

8. The retractor of claim 1, wherein said expandable structure comprises retractor arms, said drive structure being operable to expand said arms.

9. A retractor system for surgery, comprising:
a retractor body, elongated and expandable structure attached to said retractor body, said expandable structure, when expanded, having an elongated open interior space for the insertion of a surgical instrument, and drive structure operable to expand said expandable structure; and,
at least one surgical instrument, at least a portion of said surgical instrument being positionable in said open interior space.

10. The retractor system of claim 9, wherein the expandable structure is an elongated coil.

11. The retractor system of claim 10, wherein said drive structure expands and retracts the coil.

12. The retractor system of claim 11, wherein said drive structure comprises gears which engage the coil at drive surfaces.

13. The retractor system of claim 9, wherein the expandable structure comprises at least first and second portions, said drive structure moving said first and second portions radially outward relative to one another.

14. The retractor system of claim 9, wherein said drive structure is moveable within said retractor body.

15. The retractor system of claim 9, wherein said system comprises structure for engaging a K-wire, said system further comprising a K-wire.

16. The retractor system of claim 9, further comprising an access port structure for placing into said incision after removal of said expandable structure, to retain said incision in a retracted condition.

17. The retractor system of claim 9, wherein said surgical instrument is attachable to said retractor.

18. A method for retracting an incision, comprising the steps of:
providing a retractor comprising a retractor body, elongated and expandable structure attached to said retractor body, said expandable structure, when expanded, having an elongated open interior space for the insertion of a surgical instrument, and drive structure for expanding said expandable structure;
inserting said retractor into said incision; and,
operating said drive structure to expand said expandable structure to create said open interior space for inserting a portion of a surgical instrument through said open interior space of said expandable structure.
